(19) United States
(12) Patent Application Publication
(10) Pub. No.: US 2011/0112487 A1
(43) Pub. Date: May 12, 2011

(54) SYRINGE WITH INTEGRALLY FORMED PIERCING MEMBER AND LUER SLIP

(76) Inventors: Marc Andrew Koska, Nr. Uckfield (GB); Graham John Madin, Mielkendorf (DE)

(21) Appl. No.: 12/988,989

(22) PCT Filed: Apr. 21, 2009
(86) PCT No.: PCT/GB2009/001030

§ 371 Date: Jan. 5, 2011

(30) Foreign Application Priority Data
Apr. 22, 2008 (GB) 0807271.2

(51) Int. Cl. A61M 5/34

(52) U.S. Cl. 604/218

(57) ABSTRACT

A syringe for use in vaccine reconstitution or rehydration has a barrel (1) having a plunger (2) in slidable and sealing engagement therein, and a piercing member (3) formed integrally with the barrel. The piercing member (3) has a bore (10) in communication with the distal end of the barrel, and a piercing point (12) and aperture (13) at its distal end. The distal end of the piercing member (3) has an external taper which comprises a male luer part (15) compatible with an infusion set. The length of the distal end of the piercing member (3) distally of a predetermined attachment diameter of the male luer part (15) is chosen so that it cannot fit a standard metal syringe needle, as the end of the piercing member (3) engages with another part of the metal needle before the female luer part on the needle reaches the attachment diameter. The syringe is easy to use for rehydration, but cannot be used for normal injections.

The present invention relates to syringes, and in particular to syringes for use in reconstitution or rehydration.

For reconstitution of anhydrous vaccines a specific amount of diluent is used to reconstitute the dried vaccine, which is provided in a container sealed by a rubber septum. The septum is pierced by a metal needle of a syringe containing the diluent, which is then injected into the container to reconstitute the dried vaccine. Then a number of other syringes (typically ten) are used to pierce the septum again to draw up the correct amount of reconstituted vaccine for injection. The repeated piercings tend to shred the septum, which may result in leakage of vaccine, and/or possible contamination. An alternative is to pierce the septum once with metal needle of a syringe, and then leave the needle in, while detaching the syringe body, allowing further syringe bodies to be attached sequentially to draw up the correct amount of vaccine into each syringe. This alternative is fiddly and time-consuming, and also risks contamination.

Rehydration of a dehydrated person requires a regular injection of a saline or similar solution. A catheter of an infusion set is inserted into the person. The infusion set has a female luer part, that is, a slightly tapered conical bore into which fits a correspondingly tapered male projection. Saline solution is drawn up from a container into a syringe fitted with a standard metal needle which pierces the container seal. The needle is then removed from the syringe, which has a projection on the barrel adapted to fit the female luer bore on the infusion set. Once the syringe is attached to the infusion set by the luer arrangement, the saline solution can be injected. Again this procedure is somewhat fiddly and time-consuming, and carries a risk of contamination.

US-A-2002/0065490 shows a syringe for transferring a medical substance from a container with a rubber septum to a container, using a piercing member which may be formed integrally with the syringe barrel. The piercing member fits onto a standard metal syringe needle.

According to the present invention, we provide a syringe comprising a barrel having a plunger in slidable and sealing engagement therein, and a piercing member formed integrally with the barrel, the piercing member having a bore in communication with the distal end of the barrel, and a piercing point and an aperture at its distal end, the distal end of the piercing member being formed with an external taper comprising a male luer part compatible with an infusion set, the length of the distal end of the piercing member distally of a predetermined attachment diameter of the male luer part being chosen so that the end of the piercing member engages with a part of a standard metal syringe needle before a female luer part on the needle reaches the attachment diameter.

The syringe of the invention may therefore be used for rehydration purposes, to pierce a seal of a container and draw up a solution, and then to connect directly to an infusion set, using the luer connection, for injection of the solution. It has the advantages that it is easy to use, and reduces the risk of contamination. It cannot be used for normal injections, as it will not fit a standard metal syringe needle, thus reducing the risk of incorrect injections.

The syringe barrel and piercing member may be moulded in one piece, from a suitable plastics material. One such material is polypropylene, which has sufficient rigidity to make the piercing point, although the point will degrade with use. The point will not be suitable for piercing the skin of a human or animal body at all.

The taper angle of the male luer part of the piercing member is preferably about 6°, at an appropriate attachment diameter, to fit a standard infusion set.

The barrel may have at its distal end a reduced diameter portion, to which the piercing member is attached. The external diameter of the piercing member is conveniently slightly less than the reduced diameter portion of the barrel. A protective cap may then fit over the piercing member, and be retained by the reduced diameter portion of the barrel.

The plunger preferably comprises a rod carrying a head, on which is mounted a seal for engagement with the internal surface of the barrel. A projection is formed on the distal side of the plunger head, the projection fitting into the bore of the piercing member as the plunger is moved distally. The projection ensures that there is no dead space in the syringe, and enables an accurate amount of liquid to be drawn into the barrel and subsequently dispensed. This has the advantage that the syringe can also be used to reconstitution of vaccines, where it is important that an accurate amount of diluent is used.

An embodiment of the invention is illustrated, by way of example only, in the accompanying drawings, in which:

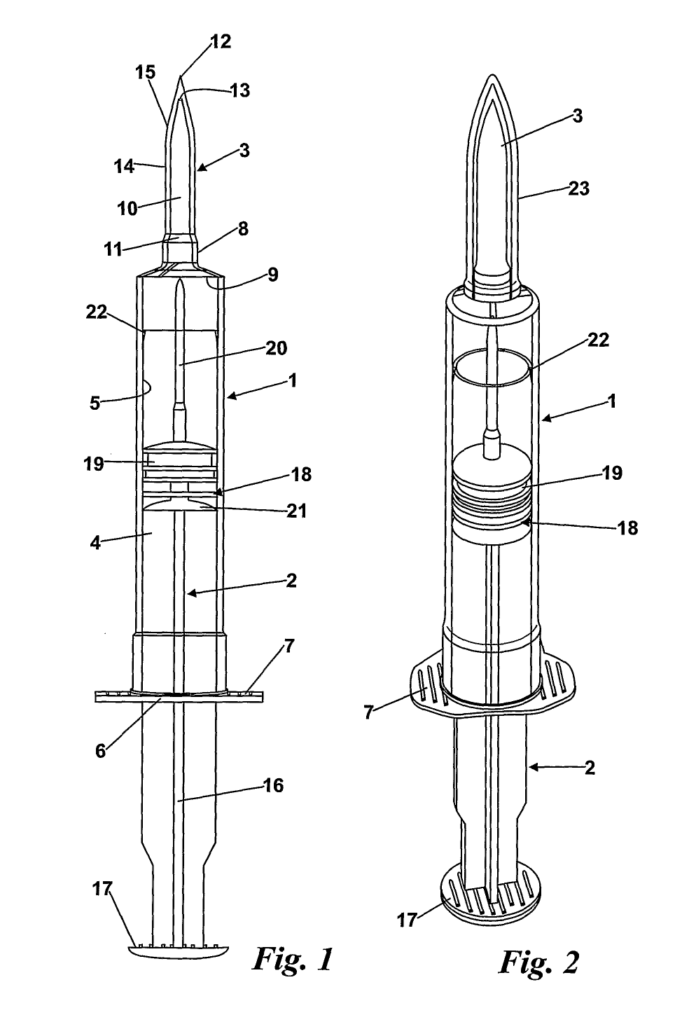

FIG. 1 shows a side view of a syringe; and

FIG. 2 is a perspective view of the syringe of FIG. 1, showing a protective cap in place.

The syringe shown in the Figures comprises a barrel 1 in which works a plunger 2, and a piercing member 3 formed integrally with the barrel 1.

The barrel 1 is generally cylindrical, having a main cylinder 4, with an inner cylindrical surface 5 in which the plunger 2 slides sealingly. The proximal end 6 of the barrel 1 is open, and is provided with a gripping flange 7. The distal end has a reduced diameter portion 8, with the step between the main cylinder 4 and the reduced diameter portion 8 forming an internal abutment shoulder 9. In a standard syringe the reduced diameter portion 8 would have an open end to define a liquid outlet, and a metal needle would be fitted onto the reduced diameter portion 8 in any suitable way. Examples of fittings are a luer slip, which holds the needle as a friction fit, a luer lock onto which the needle screws, or a permanent fixing by means of glue or the like. In the syringe of the invention, as shown, the reduced diameter portion 8 is attached instead to the piercing member 3.

The piercing member 3 is a substantially cylindrical member having a bore 10 whose proximal end 11 is in communication with the interior of the barrel 1. At its distal end the member 3 has a sharp piercing point 12 and an aperture 13 forming a liquid inlet/outlet. The external surface 14 of the distal end is formed with a cylindrical inward taper of about 6°, which forms a male luer part 15 of a standard attachment diameter adapted to fit into the female luer part of a standard infusion set (not shown). The luer connection is of the luer slip type, where the parts are held by friction. The male luer part 15 will not attach to a standard metal needle (not shown), because of the length of the point 12 distally of the luer part 15. If a standard metal needle is put on to the point 12, the distal end will engage another part of the needle hub before the male luer part 15 engages the female luer part of the needle at the attachment diameter.

The barrel 1 and piercing member 3 are moulded in one piece, from polypropylene. The polypropylene can be moulded with sufficient rigidity to make the sharp piercing point 12. The point 12 will not be suitable for piercing skin, so the syringe cannot be used for direct injection into the human or animal body. The point 12 will degrade with use, but will typically be able to pierce a rubber septum up to six times.

The plunger 2 comprises a rod 16 having a finger plate 17 at its proximal end and at its distal end a head 18 to which is mounted an elastomeric seal 19 forming a complete seal with the inner surface 5 of the barrel 1. A projection 20 is formed on the distal side of the plunger head 18. The projection 20 is substantially cylindrical, and is shaped to correspond to the internal bore of the reduced diameter portion 8 and the bore 10 of the piercing member 3. Then, with the plunger 2 fully depressed so that the seal 19 engages the abutment shoulder 9, the projection 20 is fully received in the reduced diameter portion 8 and the bore 10 of the piercing member 3. This ensures that there is no dead space, so that all liquid in the syringe will be dispensed when the plunger 2 is fully depressed. The barrel 1 may have a scale (not shown) to indicate the volume that it contains.

The head 18 of the plunger 2 may also have a locking member 21. The locking member 21 normally slides in the barrel 1 with the head 18, but if it passes over a projection 22 on the inner surface 5 at the distal end of the barrel 1, the locking member 21 engages behind the projection 22 on a withdrawal movement of the plunger 2 to lock it and prevent further withdrawal. The locking member 21 will only operate if the plunger 2 is fully depressed.

FIG. 2 shows the syringe fitted with a protective cap 23. The cap 23 is substantially the same shape as the piercing member 3, and fits over it, being retained frictionally by engagement with the reduced diameter portion 8.

For rehydration use the cap 23 is removed from the syringe, and with the plunger 2 in its rest position the piercing point 12 is used to pierce a seal such as a rubber septum of a container (not shown) holding a saline or other rehydration solution. The plunger 2 is then withdrawn to draw up solution into the barrel 1 through the opening 13 and the bore 10. The piercing member 3 is then withdrawn from the container, and the cap 23 put on it, if necessary. The syringe can then be taken to a patient who has previously been fitted with an infusion set (not shown), the cap 23 removed and the piercing member 3 attached directly to the female luer part of the infusion set. The male luer part 15 fits into the female part and is held there by friction in the usual way. The plunger 2 is then depressed to inject the solution in the barrel via the bore 10 and opening 13 to the infusion set. The syringe can be removed when the injection has taken place in readiness for the next one. The plunger 2 may be depressed several times to administer a full course. Then once the full course, of several injections at varying intervals, is complete full depression of the plunger 2 actuates the locking member 21, so that the syringe cannot be used again.

The syringe may also be used for the reconstitution of anhydrous vaccines. For this, the syringe is uncapped, and the piercing point 12 used to pierce a seal of a container of diluent, and the plunger 2 withdrawn to draw up the correct volume. The piercing point 12 is then removed from the diluent container, and used to pierce the seal of a vial of anhydrous vaccine. The diluent is dispensed into the vial by depression of the plunger 2. Due to the projection 20, an accurate amount of diluent can be dispensed, which is important for vaccine reconstitution. After dispensing the diluent, the piercing point is withdrawn, and the vaccine can be drawn up by separate standard syringes. In this case the plunger 2 of the syringe shown in the Figures will normally be fully depressed, so that the locking member 21 will be actuated to render the syringe unusable.

1-7. (canceled)

8. A syringe comprising a barrel having a plunger in slidable and sealing engagement therein, and a piercing member formed integrally with said barrel, said piercing member having a bore in communication with the distal end of said barrel, and a piercing point and an aperture at its distal end, said distal end of said piercing member being formed with an external taper comprising a male luer part compatible with an infusion set, wherein the length of said distal end of said piercing member distally of a predetermined attachment diameter of said male luer part is chosen so that said end of the piercing member engages with a part of a standard metal syringe needle before a female luer part on said needle reaches said attachment diameter.

9. A syringe as claimed in claim 8, wherein said syringe barrel and said piercing member are moulded in one piece.

10. A syringe as claimed in claim 8, wherein said taper angle of said male luer part of said piercing member is about 6°, at said predetermined attachment diameter.

11. A syringe as claimed in any claim 8, wherein said barrel has at said distal end a reduced diameter portion, to which said piercing member is attached.

12. A syringe as claimed in claim 11, wherein an external diameter of said piercing member is less than said reduced diameter portion of said barrel.

13. A syringe as claimed in claim 12, wherein a protective cap is provided to fit over said piercing member, and is retained by said reduced diameter portion of the barrel.

14. A syringe as claimed in claim 8, wherein said plunger comprises a rod carrying a head, said head having a seal mounted thereon for engagement with the internal surface of said barrel, and a distal side of said head being formed with a projection fitting into said bore of said piercing member as said plunger is moved distally.
